(19) United States
(12) Patent Application Publication
(10) Pub. No.: US 2025/0318893 A1
(43) Pub. Date: Oct. 16, 2025

(54) RETRACTABLE CORD SYSTEM

(71) Applicant: Allison KLEIN, Blacklick, OH (US)

(72) Inventor: Allison KLEIN, Blacklick, OH (US)

(21) Appl. No.: 18/635,733

(22) Filed: Apr. 15, 2024

(51) Int. Cl. A61B 50/20; A61B 5/00; H02G 11/02

(52) U.S. Cl. CPC A61B 50/20; A61B 5/742; H02G 11/02; A61B 2562/221

(57) ABSTRACT

A retractable cord system for a medical device includes a housing, a plurality of winding assemblies, and a retraction device. The plurality of winding assemblies are disposed in the housing. Each winding assembly of the plurality of winding assemblies includes a respective spool. Each spool is configured to accommodate a respective cord of a plurality of cords. The retraction device is configured to wind one or more of the plurality of cords onto the respective one or more spools and retract the one or more cords into the housing. Each winding assembly is configured to facilitate withdrawing at least a portion of the respective cord from the housing.

### FIELD OF THE INVENTION

The disclosure generally relates to a cord retraction device and, more particularly to a system for retracting cords for a patient monitor.

### BACKGROUND OF THE INVENTION

Patient monitoring devices are used to display the vital signs of a patient. Typically, a patient monitor includes several leads or cords to connect the monitor to sensors. The sensors can be attached to or interact with a patient to collect information and the monitor displays this information for medical professionals to see. The cords may include leads configured to conduct an electrical signal, tubes to convey a fluid such as air, and the like. Some examples of sensors include a blood pressure cuff, a saturation of peripheral oxygen sensor, a set of telemetry pads to sense heart rate and/or rhythm, a temperature probe, an arterial line pressure sensor, and the like. Other devices that may be connected via one or more cords include a sequential compression device, for example.

It is important that the cords and sensors are able to be easily and quickly introduced to the patient so that the vital signs of the patient can be monitored. However, due to the length and number of cords, medical personnel must devote a significant amount of time managing the cords so that the cords are easily deployed when needed. For example, if cords become twisted or tangled, medical personnel must spend time correcting this issue before the vital signs of the patient can be monitored. In another example, if any cords fall to the ground, the cords will have to be cleaned and may be a tripping hazard and extra time may be spent cleaning and managing the cords.

To address this deficiency, patient monitors typically include a series of hooks or hangers to hold the cords. In such systems, a cord may be coiled and hung from the hook or hanger. Unfortunately, coiling a cord may introduce a twist to the cord that may cause the cord to tangle. In addition, removing one cord may cause another cord to be dislodged from the hanger.

Accordingly, it is desirable to a cord management system that addresses the deficiencies of conventional systems, at least to some extent.

### SUMMARY OF THE INVENTION

It is an object of the invention to provide a cord management system that facilitates expedited use and storage which, in turn, facilitates improved patient care.

This object is achieved according to a first aspect of the invention by providing a retractable cord system for a medical device. The retractable cord system includes a housing, a plurality of winding assemblies, and a retraction device. The plurality of winding assemblies are disposed in the housing. Each winding assembly of the plurality of winding assemblies includes a respective spool. Each spool is configured to accommodate a respective cord of a plurality of cords. The retraction device is configured to wind one or more of the plurality of cords onto the respective one or more spools and retract the one or more cords into the housing. Each winding assembly is configured to facilitate withdrawing at least a portion of the respective cord from the housing.

This object is achieved according to a second aspect of the invention by providing a retractable cord device for a patient monitor. The retractable cord device includes a housing, a pressure cuff tubing winding assembly, a saturation of peripheral oxygen lead, and a heart monitor lead. The pressure cuff tubing winding assembly is disposed in the housing. The pressure cuff tubing winding assembly is configured to facilitate dispensing a pressure cuff tube from a respective spool. The pressure cuff tubing winding assembly is configured to facilitate retracting the pressure cuff tube onto the respective spool. The saturation of peripheral oxygen (SPO₂) lead winding assembly is disposed in the housing. The SPO₂ lead winding assembly is configured to facilitate dispensing a SPO₂ lead from a respective spool. The SPO₂ lead winding assembly is configured to facilitate retracting the SPO₂ lead onto the respective spool. The heart monitor lead winding assembly is disposed in the housing. The heart monitor lead winding assembly is configured to facilitate dispensing a set of heart monitor leads from one or more respective spools. The heart monitor lead winding assembly is configured to facilitate retracting the set of heart monitor leads onto the respective spools.

This and other aspects of the invention will be apparent from the figure and the embodiments described below.

### BRIEF DESCRIPTION OF THE DRAWINGS

The foregoing summary, as well as the following detailed description of the invention, will be better understood when read in conjunction with the appended figures. For the purpose of illustrating the invention, the figures demonstrate embodiments of the present invention. It should be understood, however, that the invention is not limited to the precise arrangements, examples, and instrumentalities shown.

FIG. 1 is a system diagram of a patient monitoring system in accordance with an embodiment of the invention.

FIG. 2 is a perspective view of a patient monitor and a cord management system of FIG. 1.

FIG. 3 is a perspective view of a cord management system in accordance with another embodiment.

FIG. 4 is a rear perspective view of the patient monitor and a cord management system in accordance with another embodiment.

FIG. 5 is an exploded view of a winding assembly for the cord management system of FIG. 4.

### DETAILED DESCRIPTION

In general, embodiments of a cord management system disclosed herein may be used to manage any suitable type of cord. Examples of suitable cords include electrical leads, pneumatic tubing, and the like. For the purposes of this disclosure, the terms, “cord management”, manage, and related terms refers to deploying or withdrawing one or more cords, retracting one or more cords, storing one or more cords, cleaning and/or sterilizing one or more cords, maintaining one or more cords in a secure and easily deployed manner, and the like.

It is an advantage of embodiments of the cord management system disclosed herein that the cords associated with a patient monitor may be stored in a secure and compact format in which each cord is easily deployed, and each cord is automatically retracted in response to an action of a user. In various examples, the cord management system includes one or more spools, and each spool is configured to receive one or more cords. The one or more cords may be wound onto and off the respective spool to retract and deploy, respectively, the one or more cords.

According to various examples, each spool may be controllably urged to rotate via an actuator, a manual winding device, a biasing device, or the like. As described herein, the rotation of the one or more spools facilitates retraction of the one or more cords so that the cords may be securely stored and available for deployment and use. An advantage of the cords being stored on spools is that a desired amount of the cord may be deployed and excess cord may be allowed to remain wound upon the spool. In this manner, the cord management system improves cord management and reduces potential cord tangling or damage. These and other advantages are described herein.

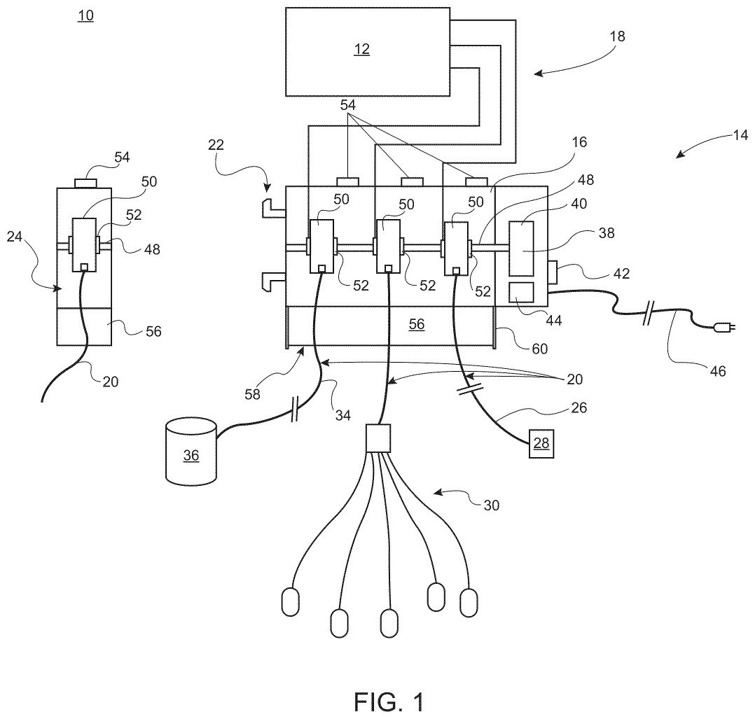

FIG. 1 is a system diagram of a patient monitoring system 10 in accordance with an embodiment of the invention. As shown in FIG. 1, the patient monitoring system 10 includes a patient monitor 12 and a cord management system 14. The patient monitor 12 includes any suitable patient monitoring device for receiving and/or displaying health related data of a patient. Particular examples of suitable patient monitors include devices manufactured by GE Healthcare such as the GE Dash 4000, Phillips such as the IntelliVue X2, and the like.

The cord management system 14 includes a retractable cord device 16, one or more patch cords 18, and one or more cords 20. The retractable cord device 16 is configured to manage the one or more cords 20. In the example shown in FIG. 1, the retractable cord device 16 is configured to manage three cords. In other examples, the retractable cord device 16 may be configured to manage any suitable number of cords, such as, one, two, three, four, five, or more. In some examples, the retractable cord device 16 may be configured to manage a fixed number of cords 20. In other examples, the retractable cord device 16 may be modular or otherwise operable to modulate the number of the cords 20 that are managed. For example, the retractable cord device 16 may include a modular latch 22 configured to engage a modular cord retractor unit 24. In the particular example shown, the modular cord retractor unit 24 is configured to manage one of the cords 20. In other examples, the modular cord retractor unit 24 may be configured to manage two or more of the cords 20. In yet other examples, the modular cord retractor unit 24 may also include the modular latch 22 operable to engage another modular cord retractor unit 24.

The one or more patch cords 18 are configured to interconnect the patient monitor 12 and the retractable cord device 16. As such, the patch cords 18 may include electrical leads suitable for conveying electrical power and/or data, pneumatic tubing suitable for conveying a pressurized fluid such as air, and the patch cords 18 may include any other cord that is suitable for use with the patient monitor 12. The one or more cords 20 may include conventional cords suitable for use with conventional vital sign sensors and medical devices. For example, the one or more cords 20 may include a pneumatic tube for a blood pressure cuff, a saturation of peripheral oxygen lead, a heart rate lead, one or more heart monitor leads, a temperature probe lead, an arterial line pressure lead, and one or more pneumatic tubes for a sequential compression device. In the example shown in FIG. 1, three cords 20 are illustrated: a lead 26 for a saturation of peripheral oxygen sensor 28, a lead 30 configured to interface with a plurality of telemetry pads 32, and a pneumatic tube 34 for a blood pressure cuff 36.

The cord management system 14 optionally includes an actuator 38. If included, the actuator 38 is configured to urge the cords 20 to be retracted into the retractable cord device 16 in response to a command by medical personnel. For example, the actuator 38 may include a motor 40 operated by a button 42 and powered via a power supply such as a battery 44 and/or a power cord 46. In response to being activated, the motor 40 configured to urge a shaft 48 to rotate. The shaft 48 is coupled to one or more spools 50 via a coupling 52. The one or more spools 50 are each operable to receive a respective cord 20. In response to rotation of spools 50 in a first direction, the respective cords 20 may be wound onto the associated spools 50 and the cords 20 may be retracted into the retractable cord device 16. In some examples, the coupling 52 between the shaft 48 and the spools 50 may be configured to allow each spool 50 to separately rotate relative to the shaft 48. In this manner, a medical professional may withdraw one of the cords 20 at a time. In addition, this feature may reduce damage to a tangled cord and/or discomfort to a patient if a medical device is attached while the cords 20 are being retracted. To allow the spools 50 to separately rotate relative to rotation of the shaft 48, the coupling 52 may include a clutch or friction fitting that is configured to provide up to a predetermined maximum amount of torque to transfer between the shaft 48 and the spool 50. This predetermined maximum amount of torque may to determined based on a calculated amount of torque required to retract a cord, empirical measurements, and the like.

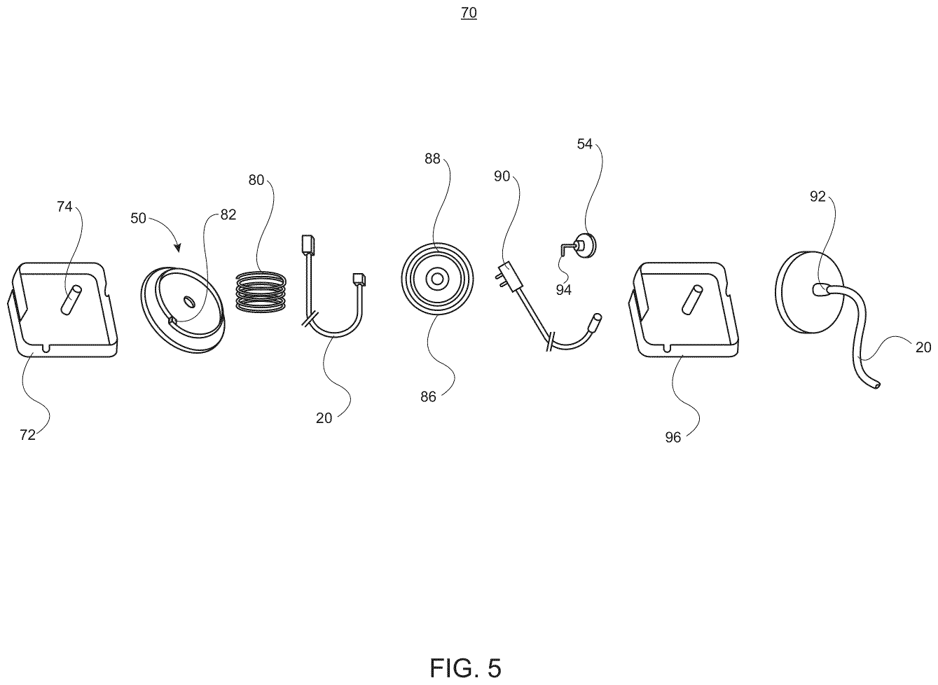

In various other examples, the retractable cord device 16 may include a button 54 associated with each spool 50 so that each spool 50 may be selectively rotated by an associated actuator. Alternatively, and as described herein, each spool 50 may include a biasing device such as a spring (see FIG. 5) and the buttons 54 may be configured to release a break to allow the associated cord 20 to be retracted (see FIG. 5).

Optionally, the cord management system 14 may include a cord wipe device 56. If included the cord wipe device 56 may be disposed on or near the retractable cord device 16. For example, as shown in FIGS. 1-4, the cord wipe device 56 may include a pair of pads 58 secured to the retractable cord device 16 via a bracket 60. The cords 20 may be disposed between the pair of pads 58. A cleaning/sanitizing solution may be applied to the pair of pads 58 to facilitate cleaning and/or sanitizing the cords 20 as the cords 20 are retracted and/or withdrawn. The pair of pads 58 may be disposable and replaced after each use, between one patient and the next, and/or at any suitable interval.

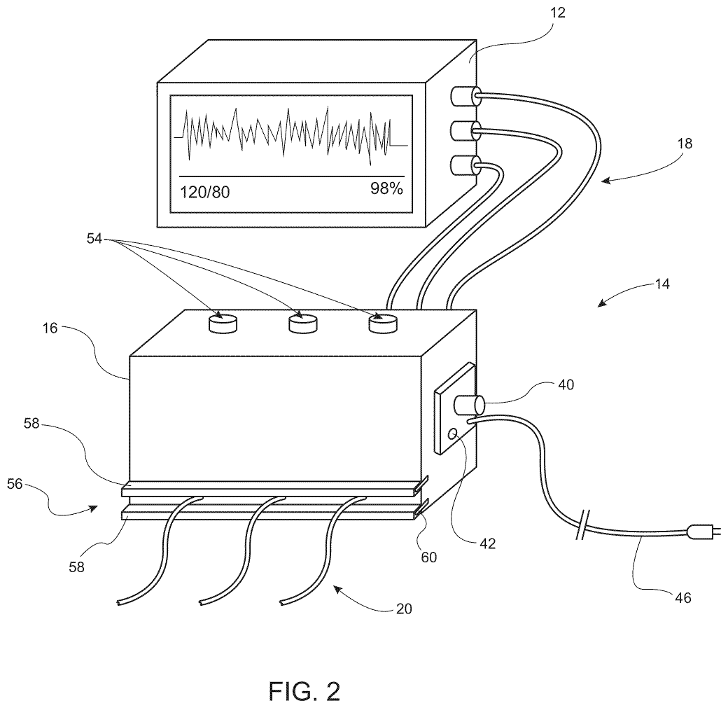

FIG. 2 is a perspective view of a patient monitor 10 and a cord management system 14 of FIG. 1. The cord management system 14 shown in FIG. 2 is similar to the cord management system 14 shown in FIG. 1 and thus, for the sake of brevity, those elements already described may not be described again. As shown in more detail in FIG. 2, the pair of pads 58 include an upper and lower wipe with the cords 20 being disposed between the pair of pads 58. Also shown in FIG. 2, the cord retraction device 16 may be disposed in close proximity to the patient monitor 12. For example, the cord retraction device 16 may be disposed at a location typically reserved for cord hangers, basket, or hooks. It is an advantage of the cord management system 14 that it provides improved cord management within substantially the same physical space as a conventional cord hanger, basket, or hooks.

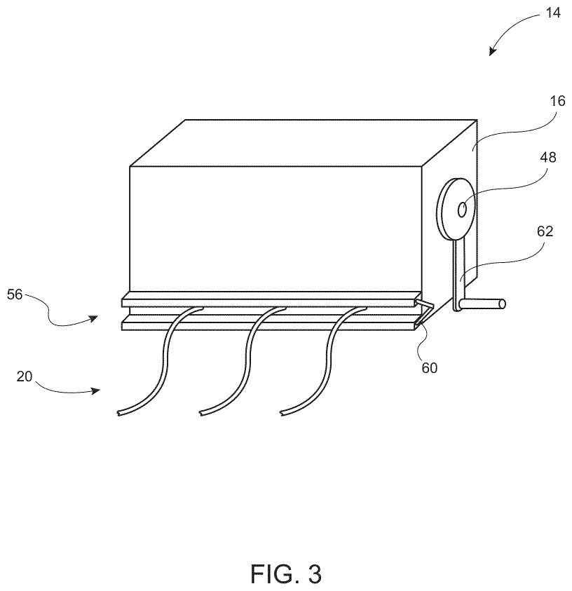

FIG. 3 is a perspective view of a cord management system 14 in accordance with another embodiment. The cord management system 14 shown in FIG. 3 is similar to the cord management system 14 shown in FIGS. 1 and 2 and thus, for the sake of brevity, those elements already described may not be described again. As shown in FIG. 3, the retractable cord device 16 includes a crank 62 configured to retract the cords 20. For example, the crank 62 may be affixed to the shaft 48. In a manner similar to the actuator 38 described herein, rotation of the crank 62 urges the shaft 48 to rotate which, in turn, urges the spools 50 to rotate and retract the cords 20.

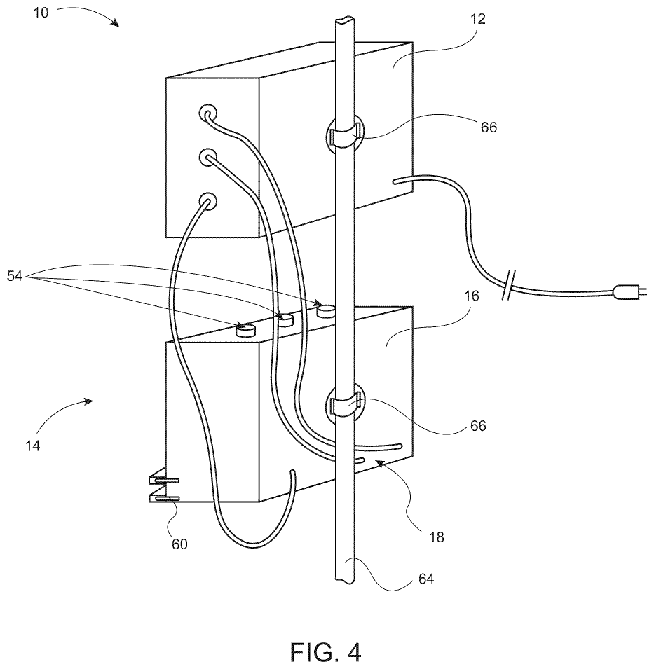

FIG. 4 is a rear perspective view of the patient monitor 12 and a cord management system 14 in accordance with another embodiment. The cord management system 14 shown in FIG. 4 is similar to the cord management system 14 shown in FIGS. 1 to 3 and thus, for the sake of brevity, those elements already described may not be described again. As shown in FIG. 4, the cord management system 14 may be mounted on a monitor stand pole 64. For example, the retractable cord device may be mounted on the monitor stand pole 64 via a mounting bracket 66. Similarly, the patient monitor 12 may be mounted to the monitor stand pole 64 via a respective mounting bracket 66.

Also shown in FIG. 4, the buttons 54 are break release buttons configured to selectively release an associated break (see FIG. 5) and allow the associated cord 20 to retract into the retractable cord device 16.

FIG. 5 is an exploded view of a winding assembly 70 for the cord management system 14 of FIG. 4. As shown in FIG. 5, the winding assembly 70 includes a first housing 72 having a shaft 74, patch cord opening 76, and cord opening 78. The shaft 74 is fixed in the first housing 72. When assembled, the spool 50 is disposed on the shaft 74. A biasing device 80 is configured to provide sufficient torque to retract the respective cord 20 into the retractable cord device 16. The biasing device 80 may include a spring, such as a helical spring, or the like. For example, in a generally well understood manner, a first end of the biasing device 80 is fixed to the shaft 74, a second end of the biasing device 80 is fixed to the spool 50, and the spool 50 is wound about the shaft 74 to generate spring tension that is utilized to retract the cord 20.

The spool 50 includes a cord connector 82 and a patch cord connector assembly 84. Depending upon the type of cord 20, the cord connector 82 and the patch cord connector assembly 84 may be configured to convey electrical signal and/or power or may be configured to convey a fluid such as air. If the cord connector 82 and the patch cord connector assembly 84 are configured to convey electrical signal and/or power the cord connector 82 may include a pin-type connector and the patch cord connector assembly 84 may include a contact disk 86 with one or more contact rings 88. Corresponding pickups 90 may be disposed on cooperative alignment with the contact rings 88 so that signals and/or power may be transmitted through the rotating spool between the patch cord 18 and the cord 20. If the cord connector 82 and the patch cord connector assembly 84 are configured to convey a fluid, the cord connector 82 may include a barb-type hose fitting and the patch cord connector assembly 84 may include a rotary pneumatic connector 92.

The winding assembly 70 further includes the button 54. When assembled, the button 54 is configured to stop or reduce rotation of the corresponding spool 50. For example, the button 54 may include an armature 94 extending through a button opening 96 in the first housing 72. The armature 94 is configured to engage the spool 50 in a biased position and disengage from the spool 50 in response to the button 54 being pressed. For example, the armature 94 and spool 50 may be configured to interact in a manner similar to a ratchet and pawl. A second housing 96 is shown in FIG. 5 to indicate that two or more winding assemblies 70 may be stacked together to fabricate the retractable cord device 16.

The invention has been described in conjunction with various embodiments herein. However, other variations to the disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality. The mere fact that certain measures are recited in mutually different dependent claims does not indicate that a combination of these measured cannot be used to advantage. The reference signs used in the claims shall not be construed as limiting the scope.

The many features and advantages of the invention are apparent from the detailed specification, and thus, it is intended by the appended claims to cover all such features and advantages of the invention which fall within the true spirits and scope of the invention. Further, since numerous modifications and variations will readily occur to those skilled in the art, it is not desired to limit the invention to the exact construction and operation illustrated and described, and accordingly, all suitable modifications and equivalents may be resorted to, falling within the scope of the invention.

What is claimed is:

1. A retractable cord system for a medical device, the system comprising:
a housing;
a plurality of winding assemblies disposed in the housing, wherein each winding assembly of the plurality of winding assemblies includes a respective spool, each spool is configured to accommodate a respective cord of a plurality of cords;
a retraction device configured to wind one or more of the plurality of cords onto the respective one or more spools and retract the one or more cords into the housing; and
wherein each winding assembly is configured to facilitate withdrawing at least a portion of the respective cord from the housing.

2. The retractable cord system according to claim 1, wherein each winding assembly includes a rotary connector.

3. The retractable cord system according to claim 2, wherein the rotary connector includes at least one conductive ring and a respective contact for each conductive ring.

4. The retractable cord system according to claim 2, wherein the rotary connector is a pneumatic rotary joint.

5. The retractable cord system according to claim 1, wherein the retraction device is a biasing device configured to bias the one or more winding assemblies to retract the one or more cords into the housing.

6. The retractable cord system according to claim 5, further comprising:
a brake configured to provide sufficient friction to prevent the biasing device from automatically retracting the one or more cords into the housing; and
a brake release to release the brake and facilitate the biasing device retracting the one or more cords into the housing.

7. The retractable cord system according to claim 5, further comprising:
a ratchet configured to allow each cord to be drawn from the housing and to prevent the biasing device from automatically retracting the one or more cords into the housing; and
a ratchet release to release the ratchet and facilitate the biasing device retracting the one or more cords into the housing.

8. The retractable cord system according to claim 5, wherein the biasing device is a helical spring.

9. The retractable cord system according to claim 1, wherein the retraction device is a manual winder configured to facilitate an operator to retract the one or more cords into the housing.

10. The retractable cord system according to claim 1, wherein the retraction device is an actuator that is controllable by an operator and, when activated, the actuator is configured to retract the one or more cords into the housing.

11. The retractable cord system according to claim 1, further comprising a respective patch cord for each winding assembly, each patch cord being configured to connect a respective cord to the medical device.

12. The retractable cord system according to claim 1, wherein the medical device is a patient monitor.

13. The retractable cord system according to claim 1, wherein each cord of the plurality of cords is selected from the group consisting of a pneumatic tube for a blood pressure cuff, a saturation of peripheral oxygen lead, a heart rate lead, a heart monitor lead, a temperature probe lead, an arterial line pressure lead, and a pneumatic tube for a sequential compression device.

14. The retractable cord system according to claim 13, wherein the heart monitor lead is configured to interface with a plurality of telemetry pads.

15. The retractable cord system according to claim 1, further comprising a wipe disposed at a cord outlet of each winding assembly, the wipe being configured to clean a portion of the respective cord.

16. The retractable cord system according to claim 1, further comprising a latch to affix a modular housing to the retractable cord system, the modular housing having an additional winding assembly.

17. A retractable cord device for a patient monitor, the device comprising:
a housing;
a pressure cuff tubing winding assembly disposed in the housing, the pressure cuff tubing winding assembly being configured to facilitate dispensing a pressure cuff tube from a respective spool and the pressure cuff tubing winding assembly being configured to facilitate retracting the pressure cuff tube onto the respective spool;
a saturation of peripheral oxygen (SPO₂) lead winding assembly disposed in the housing, the SPO₂ lead winding assembly being configured to facilitate dispensing a SPO₂ lead from a respective spool and the SPO₂ lead winding assembly being configured to facilitate retracting the SPO₂ lead onto the respective spool; and
a heart monitor lead winding assembly disposed in the housing, the heart monitor lead winding assembly being configured to facilitate dispensing a set of heart monitor leads from one or more respective spools and the heart monitor lead winding assembly being configured to facilitate retracting the set of heart monitor leads onto the respective spools.

18. The device according to claim 17, wherein each winding assembly includes a rotary connector, wherein the rotary connector is selected from a group consisting of an electrical rotary connector and a pneumatic rotary connector, the electric rotary connector includes at least one conductive ring and a respective contact for each conductive ring and the pneumatic rotary connector is a pneumatic rotary joint.

19. The device according to claim 17, further comprising a respective patch cord for each winding assembly, each patch cord being configured to connect a respective cord to the medical device.

20. The device according to claim 17, further comprising a wipe disposed at a cord outlet of each winding assembly, the wipe being configured to clean a portion of the respective cord.

21. The device according to claim 17, further comprising a latch to affix a modular housing to the device, the modular housing having an additional winding assembly.
